(19) United States
(12) Patent Application Publication
(10) Pub. No.: US 2014/0156027 A1
(43) Pub. Date: Jun. 5, 2014

(54) PROSTHETIC FOOT

(71) Applicant: Ability Dynamics LLC, Tempe, AZ (US)

(72) Inventors: Keith B. Smith, Gilbert, AZ (US); Gene Parker, Mesa, AZ (US)

(21) Appl. No.: 14/175,591

(22) Filed: Feb. 7, 2014

Related U.S. Application Data
(63) Continuation-in-part of application No. 13/568,535, filed on Aug. 7, 2012.

(51) Int. Cl. A61F 2/66

(52) U.S. Cl. CPC A61F 2/66
USPC 623/54; 623/55

(57) ABSTRACT

A prosthetic foot may comprise a resilient member comprising an outer arced surface, a toe piece, a mid-stance support, and a heel support. The toe piece may be coupled to an anterior portion of the outer arced surface of the resilient member. The mid-stance support may be removably coupled to a middle portion of the outer arced surface of the resilient member. The heel support coupled to a posterior portion of the outer arced surface of the resilient member.

### CROSS-REFERENCES TO RELATED APPLICATIONS

This application is a continuation-in-part of prior U.S. patent application Ser. No. 13/568,535, filed Aug. 7, 2012, and incorporates the disclosure of such application by reference.

### BACKGROUND OF THE INVENTION

Prosthetic feet are well known in the art. In use, such prosthetic feet typically do not replicate the action of a real foot and may generate “kickback” or “kickforward” reactions that increase the risk of injury to an amputee utilizing the foot. Kickback is motion created by the prosthetic foot in a backward direction during the walking cycle. Kickforward is motion created by the prosthetic foot in a forward direction during the waking cycle. Either motion may create instability for user if expanding or restricting the intended motion. Further, many prior art prosthetic feet generate vibrations that may travel through a user's leg and cause discomfort.

For an amputee, loosing bipedality may produce an involuntary anterior lean or shift, forcing a constant imbalance or rebalance of posture. The amputee no longer possesses voluntary muscle control on his involved side due to the severance of the primary flexor and extensor muscles. The primary anterior muscle responsible for dorsiflexion (sagittal plane motion) is the anterior tibialis. Dorsiflexion is the voluntary ankle motion that elevates the foot upwards, or towards the midline of the body. The primary posterior muscle responsible for plantarflexion is the gastro-soleus complex. It is a combination of two muscles working in conjunction: the gastrocnemius and the soleus. Plantarflexion is the voluntary ankle motion that depresses the foot downwards, or away from the midline of the body.

Additionally, users of prosthetic feet often use their prosthetic for a number of applications. For example, a user may use the prosthesis for standing, walking, running and for various other athletic endeavors.

Therefore, it is desirable to have a prosthetic foot configured to promote increased muscle activity and promote increased stability for amputees, and it is desirable to provide an improved prosthetic foot which would better replicate the action of a true foot. Furthermore, it is desirable to provide an improved prosthetic foot which minimizes or eliminates “kickback” forces when the foot is utilized to walk over a door jamb or other raised profile object on a floor or on the ground, as well as reduce vibrations. Still further, it is desirable to provide a prosthetic foot that functions for multiple applications and in different situations for the user.

### SUMMARY OF THE INVENTION

An exemplary prosthetic foot may comprise a resilient bottom member having an anterior bottom end and a posterior bottom end, a resilient top member having an anterior top end and a posterior top end, wherein the anterior top end is connected to the anterior bottom end of the resilient bottom member, and wherein the resilient top member is positioned over the resilient bottom member and directed towards the posterior of the prosthetic foot, and an elastomeric bumper member comprising a tapered surface configured to contact the resilient bottom member and attached to an underside of the posterior top end of the resilient top member, wherein the bumper member is vertically oriented with respect to the prosthetic foot.

Furthermore, in another embodiment, a prosthetic foot may comprise a resilient bottom member having a first bottom end and a second bottom end, a resilient top member having a first top end and a second top end, wherein the first top end is connected to the first bottom end of the resilient bottom member, and wherein the resilient top member is positioned over the resilient bottom member and directed towards the back of the prosthetic foot, and a toe pad. The toe pad may comprise at least one spacer coupled to, and creating space between, the first bottom end of the bottom member and the first top end of the top member, and an adhesive bonding the first bottom end of the bottom member and the first top end of the top member, wherein the adhesive is commingled with the at least one spacer between the first bottom end and the first top end.

Furthermore, in another embodiment, a prosthetic foot may comprise a resilient member, a mid-stance support, and a heel support. The resilient member may comprise an anterior end, a middle portion, and a posterior end. The mid-stance support may be coupled to a lower surface of the middle portion of the resilient member. The heel support may be coupled to a lower surface of the posterior portion.

Furthermore, in another embodiment a prosthetic foot may comprise a resilient member comprising an outer arced surface, a toe pad, a mid-stance support, and a heel support. The toe pad may be coupled to an anterior portion of the outer arced surface of the resilient member. The mid-stance support may be removably coupled to a middle portion of the outer arced surface of the resilient member. The heel support may be coupled to a posterior portion of the outer arced surface of the resilient member. The design variables of the mid-stance, toe-pad and heel support may be adjustable to accommodate the user's wishes.

### BRIEF DESCRIPTION OF THE DRAWINGS

A more complete understanding of the present invention may be derived by referring to the detailed description and claims when considered in connection with the following illustrative figures. In the following figures, like reference numbers refer to similar elements and steps throughout the figures.

FIGS. 1A and 1B are perspective views illustrating a prosthetic foot constructed in accordance with various embodiments;

FIG. 2 is a rear view further illustrating the prosthetic foot of FIGS. 1A and 1B;

FIG. 3 is a side view further illustrating the prosthetic foot of FIGS. 1A and 1B;

FIGS. 4A and 4B are perspective views illustrating a prosthetic foot comprising a toe wrap;

FIGS. 5A-5C are side views illustrating various embodiments of a damper bar configuration;

FIG. 6 is a side view illustrating an exemplary prosthetic foot for use by an above-knee amputee;

FIG. 7 is a side view illustrating an exemplary prosthetic foot for use by a below-knee amputee;

FIG. 8 is a side view illustrating a prosthetic foot in accordance with various embodiments with a mid-stance support; and

FIG. 9, is a side view illustrating a prosthetic foot in accordance with various embodiments with the mid-stance support removed;

FIGS. 10A-D are views illustrating a mid-stance support in accordance with various embodiments of the present invention;

FIGS. 11A and 11B are views illustrating a toe piece in accordance with various embodiments of the present invention;

FIG. 12 is a side view illustrating a mid-stance support in accordance with various embodiments of the present invention;

FIG. 13 is a side view illustrating a prosthetic foot in accordance with various embodiments with a mid-stance support;

FIG. 14 is a side view illustrating a prosthetic foot in accordance with various embodiments with a mid-stance support;

FIGS. 15A and 15B are views illustrating a heel support in accordance with various embodiments of the present invention; and

FIGS. 16A and 16B are views illustrating a heel support and insert in accordance with various embodiments of the present invention.

Elements and steps in the figures are illustrated for simplicity and clarity and have not necessarily been rendered according to any particular sequence. For example, steps that may be performed concurrently or in a different order are illustrated in the figures to help to improve understanding of embodiments of the present invention.

### DETAILED DESCRIPTION OF EXEMPLARY EMBODIMENTS

The present invention may be described in terms of functional block components and various processing steps. Such functional blocks may be realized by any number of components configured to perform the specified functions and achieve the various results. For example, the present invention may include a prosthetic foot for above and below knee amputees. In addition, the present invention may be practiced in conjunction with any number of materials and methods of manufacture and the system described is merely one exemplary application for the invention.

Briefly, in accordance with exemplary embodiments, a prosthetic foot is illustrated which comprises a more natural motion and response of the foot occurs during movement. In particular, the movement of the exemplary prosthetic foot replicates the natural flex of a foot and supplies continuous energy to a person when striding from heel to toe. The prosthetic foot also provides for adjustability of the foot in accordance with the activity being undertaken by the user.

In an exemplary embodiment a prosthetic foot stores energy during the gait cycle and transfers the energy in order to “put a spring in your step.” The gait cycle, and specifically the stance phase, includes a heel-strike phase, a mid-stance phase, and a toe-off phase. The heel-strike phase begins when the heel of the foot touches the ground, and includes the loading response on the foot. The mid-stance phase is when the foot is flat on the ground and the body's center of gravity is over the foot. The toe-off phase is the finish of the stance phase and ends when the tip of the foot is the only portion in contact with the ground, and the load is entirely on the toe.

The roll through of a prosthetic foot is defined in the gait cycle as the process from the heel-strike phase to the mid-stance phase to the toe-off phase, where the foot is no longer in contact with the ground. As the user moves through the gait cycle the tibia portion of the leg, or that section of the leg defined below the knee, rotates through in relation to the ground. If the mid-stance phase is defined as the lower leg at 90 degrees to the ground, then looking at the left side of an individual, the angle of the lower leg at the heel-strike phase may occur at approximately 65 degrees and the angle of the lower leg at the toe-off phase may occur at approximately 110 degrees. The rotation of the lower leg on the theoretical ankle is notated as tibial progression or lower leg progression during the stance phase.

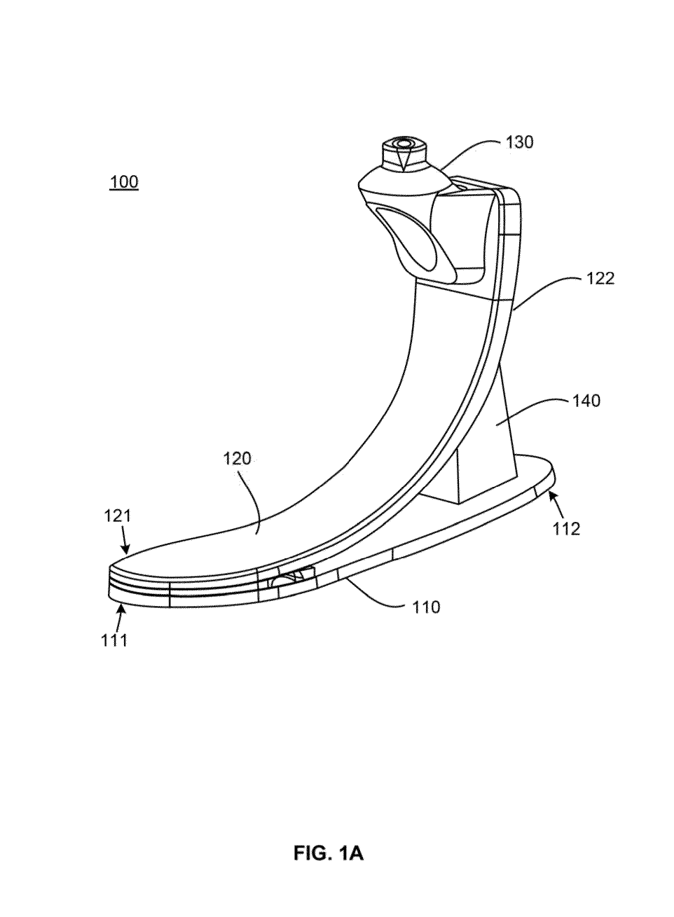

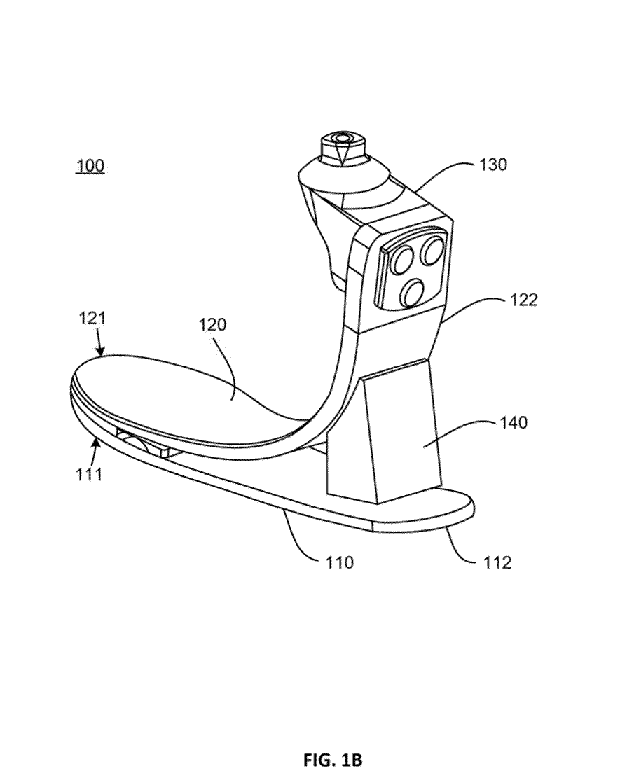

In accordance with various embodiments and with reference to FIGS. 1A and 1B, a prosthetic foot 100 may comprise a resilient bottom member 110, a resilient top member 120, a connection point 130 attached to the top member 120 and configured for attachment to a user, and a bumper member 140. The resilient bottom member 110 may comprise an anterior bottom end 111 and a posterior bottom end 112. The resilient top member 120 may comprise an anterior top end 121 and a posterior top end 122. Further, the anterior top end 121 of the resilient top member 120 may be connected to the anterior bottom end 111 of the resilient bottom member 110, while the resilient top member 120 may be positioned over the resilient bottom member 120 and directed towards the posterior of the prosthetic foot 100.

Further, in various embodiments, the prosthetic foot 100 may comprise an elastomeric bumper member 140 having a tapered surface configured to contact the resilient bottom member 110 and attached to an underside of the posterior top end 122 of the resilient top member 120. The bumper member 140 may be vertically oriented with respect to the prosthetic foot 100. The bumper member 140 may act as a heel shock for absorbing force on the downward strike during the user's stride.

In various embodiments, the bumper member 140 may comprise an elastomeric material. In one embodiment, the elastomeric material has about 80% or greater energy return. In another embodiment, the elastomeric material has about 90% or greater energy return. The bumper member 140 may be designed to behave similar to a non-linear spring, thereby allowing larger deflection of the posterior toe end 122 during the heel strike. The progressive “spring rate” may lead to a soft heel strike but does not deflect too far as the bumper member 140 compresses. One benefit of the bumper 140 is being relatively lightweight in comparison to a prosthetic foot with coiled springs.

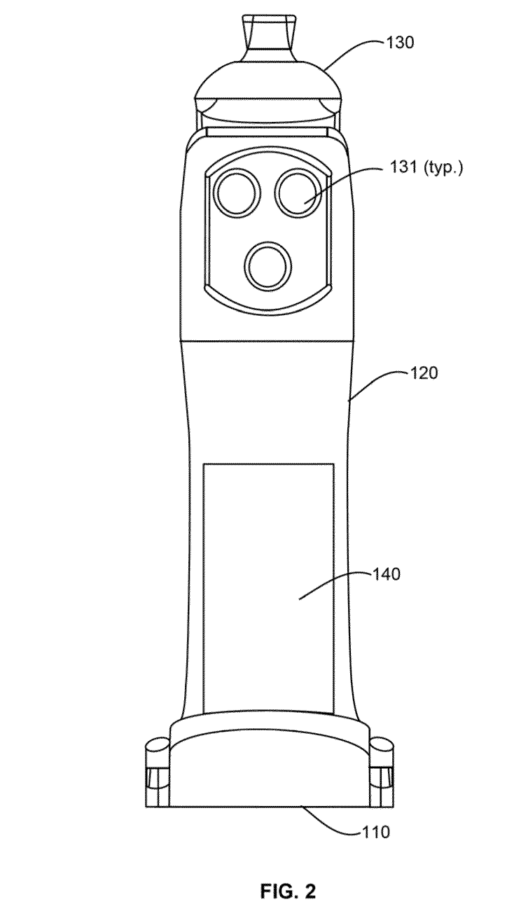

The bumper member 140 may be located posterior to vertical axis of the connection point 130. The bumper member 140 may be attached to the underside of the top member 120 in various manners. For example and with reference to FIG. 2, the bumper member 140 may be fixedly attached using adhesive or fasteners, such as screws. In another example, the bumper member 140 may be detachable using fasteners for replacement purposes. Moreover, in other embodiments, the bumper member 140 may be attached to various locations on the underside of the top member 120 or topside of the bottom member 110. In various embodiments, the prosthetic foot 100 in a static mode has a gap between the bumper member 140 and the bottom member 110. For example, a gap of about 1/10 inch may be present between the bumper member 140 and the bottom member 110. In other various methods, the bumper member 140 may be in contact with both the top member 120 and the bottom member 110 when the prosthetic foot 100 is in a static position. The lack of a gap results in the bumper member 140 being continuously compressed during the gait cycle, though the bumper member 140 is a compression member and not a tension member since the bumper member 140 is only attached to either the top member 120 or the bottom member 110.

The bumper member 140 may be constructed in many shapes. In various embodiments, the detached portion of the bumper member 140 may have a conical, rectangular, or pyramid shape. The tapered surface of the bumper member 140 may terminate in an apex or hemispherical shape, and the apex may be configured to contact the bottom member 110 in response to deflection of the prosthetic foot 100. Moreover, in various embodiments, the bumper member 140 may terminate in multiple points. The tapered bumper member 140 facilitates a damping of vibration and sound generated during heel strike or release. Furthermore, in various embodiments the extruding portion of the bumper member 140 may be any shape that is non-flat surface. Further, a non-flat surface enhances lateral flexibility if the heel strike is not vertical.

The prosthetic foot 100 may be adjusted to accommodate a user in part by adjusting characteristics of the bumper member 140. For example, in various embodiments, the durometer of the bumper member 140 may be increased for users with more heel strike force, which may be caused by additional weight or dynamic activity. A heavier user may be better-suited using a bumper member with a large cross-sectional area compared to a lighter user using a bumper member with a small cross-sectional area.

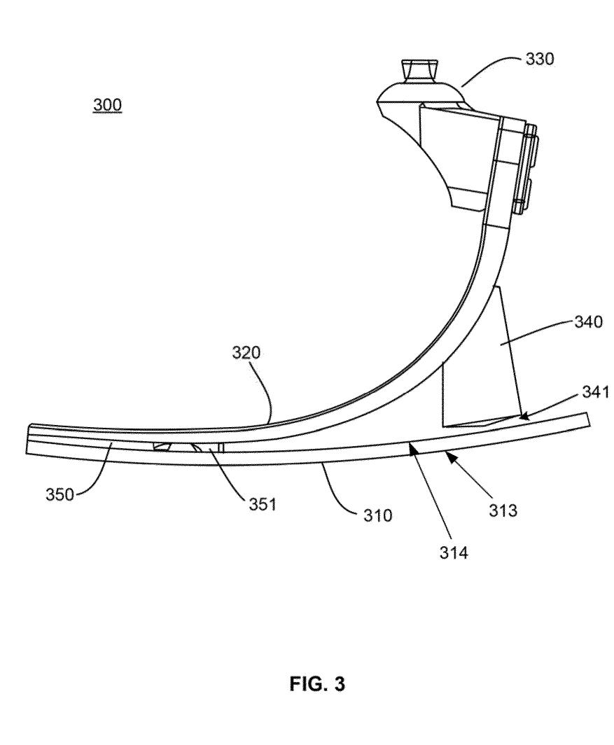

In accordance with various embodiments and with reference to FIG. 3, a prosthetic foot 300 may comprise a resilient bottom member 310, a resilient top member 320, a connection point 330 attached to the top member and configured for attachment to a user, and a toe pad 350 coupled to the top surface of the bottom member 310 at a first bottom end and coupled to the bottom surface of the top member 320 at a first top end. Also, in various embodiments, prosthetic foot 300 may further comprise a bumper member 340. In various embodiments, the toe pad 350 may comprise at least one spacer and an adhesive bonding the top surface of the bottom member 310 and the bottom surface of the top member 320. For example, the anterior quarter of the bottom member 310 may be adhesively connected to the top member 320. In various embodiments, adhesive may be used to connect 23-27% of the top surface area of the bottom member 310 to the top member 320. Further, in various embodiments, adhesive may be used to connect approximately ⅓ of the top surface area of the bottom member 310 to the top member 320.

In various embodiments, the toe pad 350 has approximately constant thickness. In other various embodiments, the toe pad 350 may have a thickness that tapers towards the front edge of the prosthetic foot 300. In other words, the toe pad 350 closer to the heel may be thicker than the toe pad 350 closer to the toe. Further, the adhesive bonding of the toe pad 350 may produce distributed stresses. In accordance with various embodiments, the adhesive may have a higher modulus of elasticity in contrast to the elastomer of the toe pad. Though other modulus values are contemplated, and various moduli may be used as well, a stiffer adhesive is preferred compared to a flexible adhesive.

The spacer of the toe pad 350 creates a space between the top surface of the bottom member 310 and the bottom surface of the top member 320. The adhesive may be commingled with the spacer between the top surface of the bottom member 310 and the toe pad 350 and also between the bottom surface of the top member 320 and the toe pad 350. In various embodiments, the space created by the spacer may be non-compressed space for the placement of the adhesive. In other words, the spacer may create a void between the top member 320 and the bottom member 310 and the void may be filled with the adhesive for bonding. The inclusion of the toe pad 350 may reduce the stress applied to the adhesive bond during the gait cycle. In various embodiments, the spacer may be elastomeric stand-offs, such as dots, ribs, or other patterns to create the desired spacing. Moreover, in various embodiments, the spacer is a single piece of connected stand-offs. The single piece spacer facilitates easier alignment during the manufacturing process and may provide a more uniform stand-off pattern compared to multiple stand-off spacers.

The toe pad 350 may comprise an adhesive composite with spacers. In various embodiments of the prosthetic foot 300, the spacer is an aggregate material combined with the adhesive to form the adhesive composite. In various embodiments, the adhesive composite includes adhesive and microspheres. The microspheres may create the spacing between the top and bottom members 320, 310.

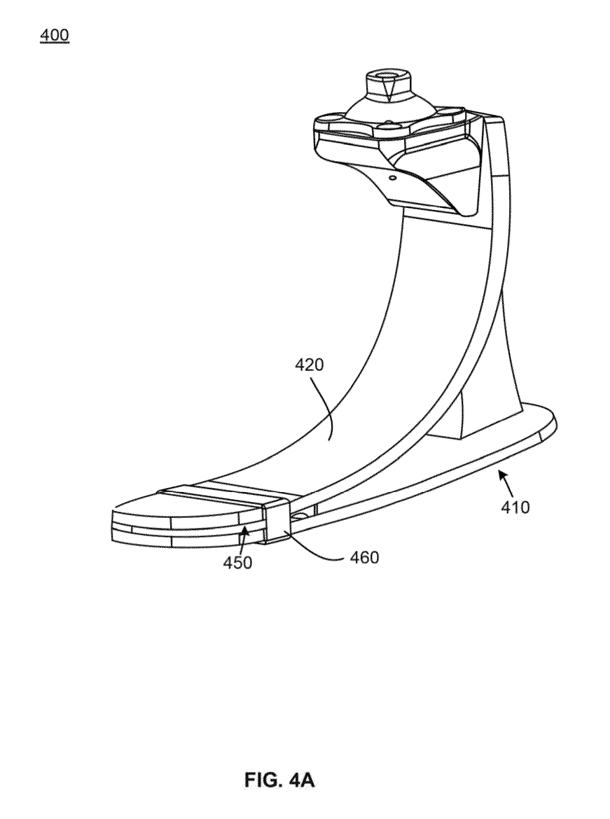

Additionally, in various embodiments and with reference to FIGS. 4A and 4B, a prosthetic foot 400 may comprise a bottom member 410, a top member 420, a toe pad 450, and a toe wrap 460 bonded around the top and bottom of the bonded bottom and top members 410, 420. The toe wrap 460 may be made out of a fiber material. The toe wrap material may also be a fiber weave with an elastomeric material. For example, the toe wrap may be a Kevlar or nylon material belt that is approximately less than a 1/10th of an inch in thickness. The toe wrap 460 may be configured to provide a secondary hold in case the adhesive bond of the toe pad 450 between the top and bottom members breaks. Also, the toe wrap 460 may strengthen the attachment between the bottom and top members 410, 420 during tension.

Moreover, in various embodiments and with renewed reference to FIG. 3, the prosthetic foot 300 may comprise a damper bar 351 configured to attach to an underside of the resilient top member 320 and contact the resilient bottom member 310. The damper bar 351 may be configured to arrest the upward motion of bottom member 310 after toe-off and also arrest the rotational energy during the gait cycle. The arrested motion creates a slower velocity and less motion at the point of contact of the damper bar 351. Without the damper bar, the bottom member 310 may slap against the bumper member 340 during the stride, resulting in vibration traveling up the leg of the user.

In various embodiments, the damper bar 351 may be located near the posterior edge of the toe pad 350. As an example, the damper bar 351 may be spaced ½ inch away from the posterior edge of the toe pad 350. In another example, the damper bar 351 may be located in the anterior portion of the bottom member 310. Further, the damper bar 351 may comprise a length of approximately a ½ inch, with the length measured from anterior to posterior of the bottom member 310. In various embodiments, the width of the damper bar 351 may be as approximately the same width as the attached top member 320. However, the damper bar 351 may also be less than the full width of the attached top member 320. Furthermore, in various embodiments, the contacting surface of the damper bar 351 may be flat. In alternative embodiments, the contacting surface of the damper bar 351 may be tapered to an apex. The contacting surface may be configured to reduce vibration and sounds caused from the contact of the non-connected bottom member 310 with the damper bar 351 during the gait cycle. Furthermore, in various embodiments, the contacting surface of the damper bar 351 may be various shapes other than flat, such as a preloaded taper.

In various embodiments, the damper bar 351 is connected to the toe pad 350, or is formed as part of the toe pad 350. One advantage of having the toe pad 350 and damper bar 351 as a single piece is for easier alignment during manufacturing of the prosthetic foot 300.

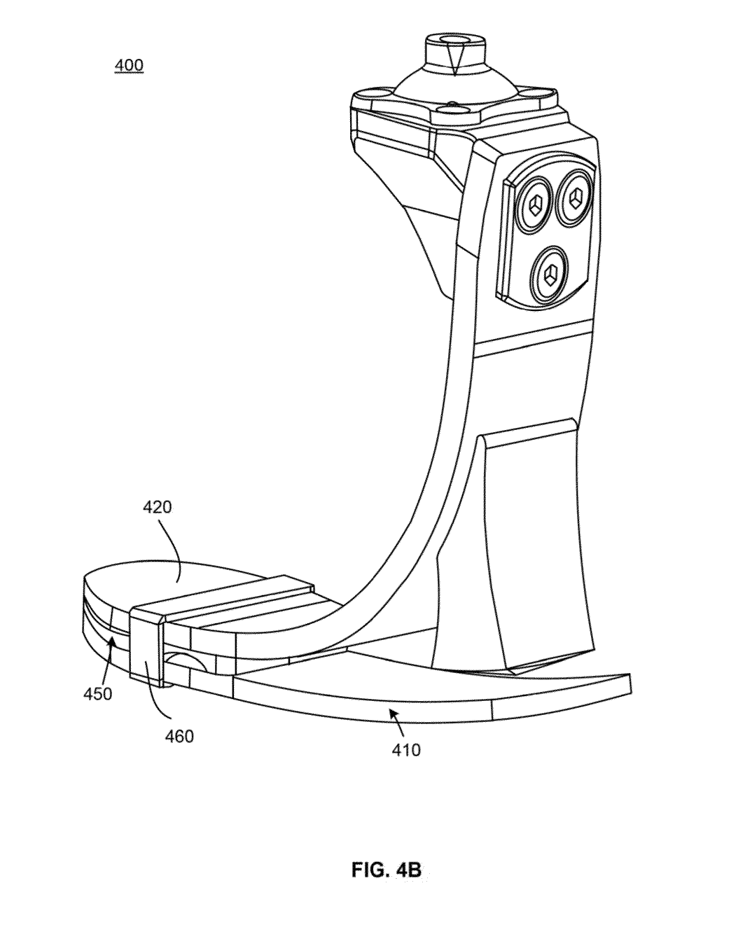

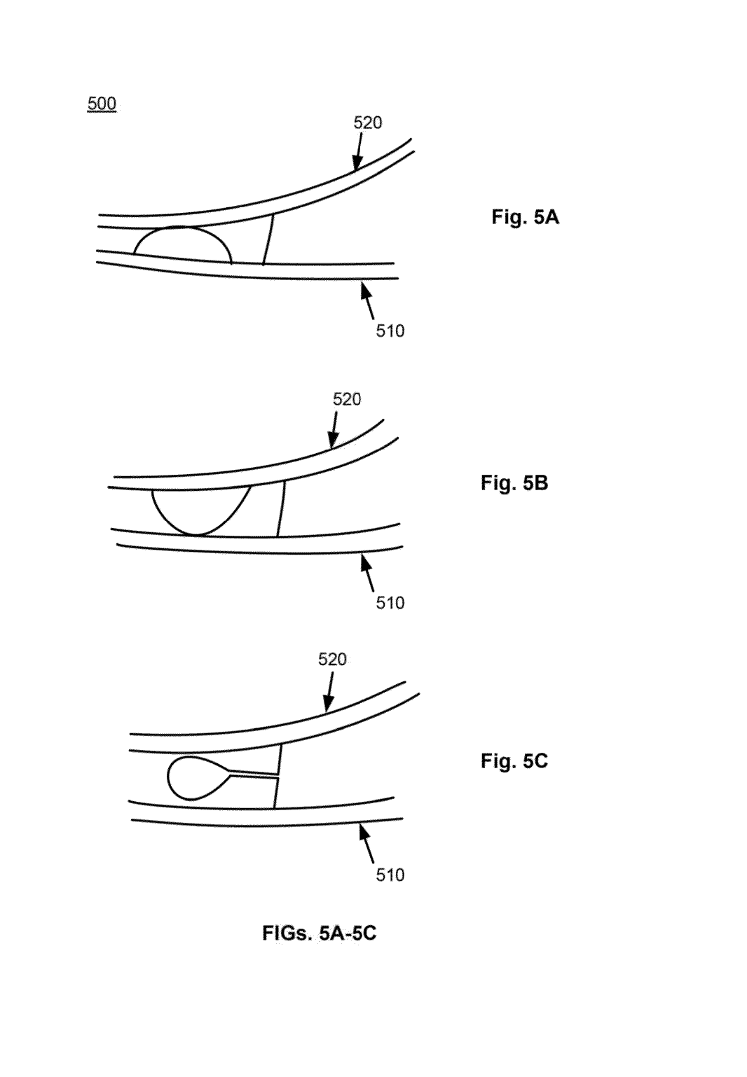

The damper bar 351 may be minimally load-bearing, whereas the bumper member 340 may be the primary load-bearing component. In various embodiments, the bumper member 340 may be located about four to five times farther back from the fulcrum point of the toe pad 350 in comparison to the damper bar 351. Furthermore, in various embodiments and with reference to FIGS. 5A-5C, a damper bar may be attached to the prosthetic foot in various configurations. For example, FIG. 5A illustrates a damper bar 551 attached to a top member 520, whereas FIG. 5B illustrates a damper bar 551 attached to a bottom member 510. In another example, FIG. 5C illustrates a damper bar 551 attached to both the bottom member 510 and the top member 520, where the damper bar 551 is divided such that the top and bottom member may separate and still arrest motion of the prosthetic foot.

Moreover and with renewed reference to FIGS. 1A and 1B, the top member 120, bottom member 110, and bumper member 140 transfer energy between themselves in a natural, true foot manner. The loading response during the heel strike phase compresses bumper member 140 and top member 120, which in turn passes energy into, and causes a deflection of, a rear portion of bottom member 110. Energy is transferred towards the front of prosthetic foot 100 during the mid-stance phase. Furthermore, an upward deflection of at least one of bottom member 110 and top member 120 stores energy during the transition from the mid-stance phase to the toe-off phase of the gait cycle. In an exemplary embodiment, about 90% or more of the heel strike loading energy is stored and transferred to top member 120 for assisting the toe-off phase. In another exemplary embodiment, about 95% or more of the heel strike loading energy is stored and transferred to top member 120 for assisting the toe-off phase. In yet another exemplary embodiment, about 98% or more of the heel strike loading energy is stored and transferred to top member 120 for assisting the toe-off phase. Prosthetic foot 100 may be designed to release the stored energy during the toe-off phase and assist in propelling the user in a forward direction.

In an exemplary embodiment and with renewed reference to FIG. 3, resilient bottom member 310 includes a bottom surface 313 and an upper surface 314. Resilient bumper member 340 includes a contact surface 341. When prosthetic foot 300 is compressed, resilient top member 320 and bumper member 340 are compressed and displaced downwardly toward resilient bottom member 310.

With respect to the walking motion, the prosthetic foot is configured to increase the surface-to-foot contact through the gait cycle. The increased surface contact allows for a smoother gait cycle, and increases stability in comparison to the typical prior art prosthetics. In exemplary embodiments, the underside of bottom member has different contours that provide increased surface contact for different types of uses.

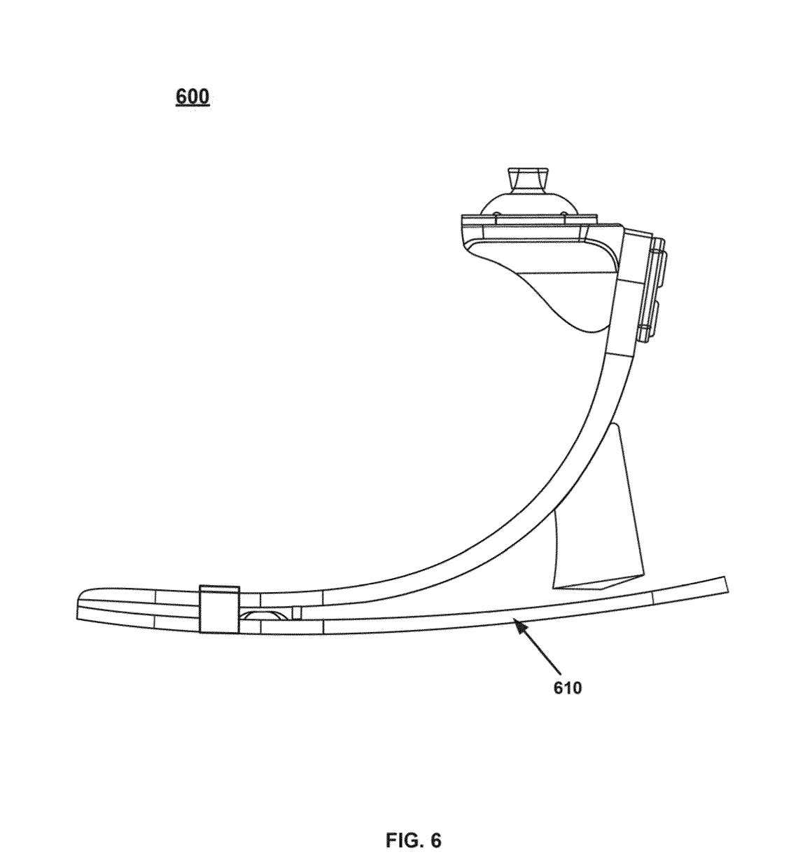

The bottom member of the prosthetic foot may have various shapes depending on desired use. The desired use may include prosthetic feet for above-knee amputees or prosthetic feet for below-knee amputees. In various embodiments and with reference to FIG. 6, a prosthetic foot 600 for above-knee amputees comprises a bottom member 610 having a curved bottom with no inflection point. In various embodiments, the bottom member 610 has a constant arc due to single radius forming the partial curve of the bottom member. In other various embodiments, the curve of the bottom member 610 may be designed as a spline of variable radii. The curve of bottom member 610 in above-knee prosthetic foot 600 facilitates keeping an artificial knee stable because the forces substantially restrict the knee from bending. The curved bottom member 610 enables a rocking motion even if the artificial knee is hyper-extended.

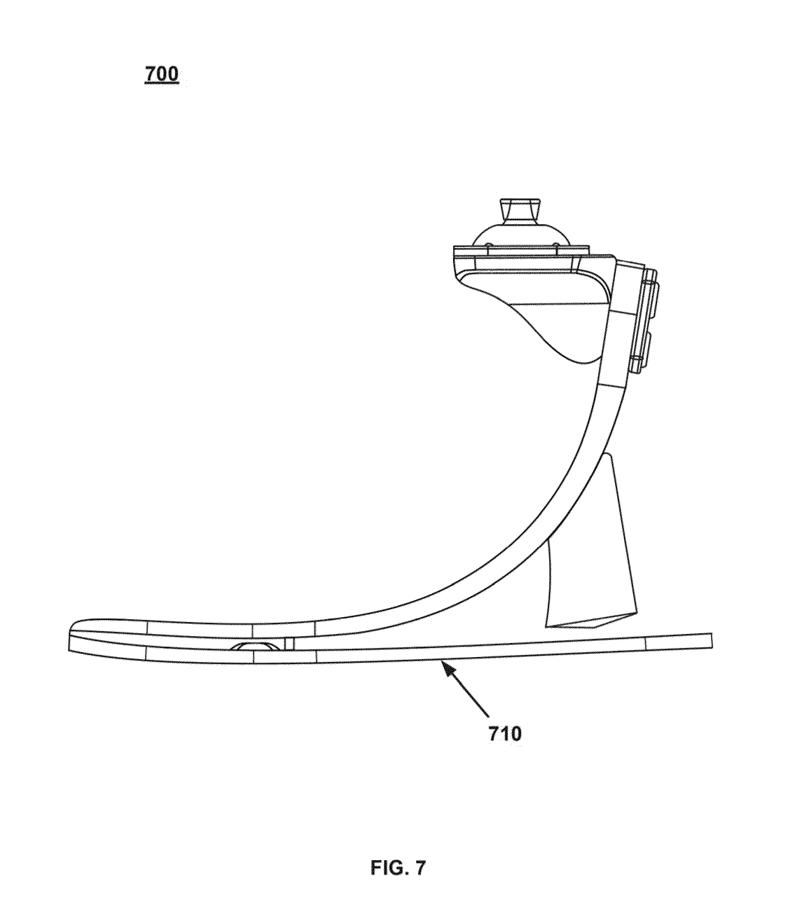

Similarly, in various embodiments and with reference to FIG. 7, a prosthetic foot 700 for below-knee amputees comprises a bottom member 710 having a partially curved portion in the anterior of the bottom member 710 and a substantially linear portion in the posterior portion of the bottom member 710. Similar to above-knee prosthetic foot 600, the anterior portion of bottom member 710 may have a constant arc due to single radius forming the partial curve. In various embodiments, the anterior portion of bottom member 710 may have a curve designed as a spline of variable radii. In accordance with various embodiments, the posterior portion of bottom member 710 may be substantially straight and tangent to the anterior portion such that bottom member 710 does not have an inflection point. A straight posterior portion and a curved anterior portion of bottom member 710 in below-knee prosthetic foot 700 facilitates rotation of the tibia progressing the natural rotation of the knee forward and preventing hyper-extension of the knee.

In various embodiments, a prosthetic foot generally has a forward section, a middle section, and a rear section. A prosthetic foot with the ability to change the design variables of all three sections (forward, middle and rear) and made of an elastomeric material, would allow the prosthetist, or end user to adjust the characteristics of the foot to meet the changing needs of the individual. For example, a user may initially want a stable, firm feel to the prosthetic foot, for use mostly while standing. However, as the user becomes stronger or more active, the user may want a foot with better roll through characteristics. The opposite may also be true. If a user becomes weaker or less active do to age or other debilitating circumstances, the user may want a more stable foot rather than a foot with high roll through characteristics. Accordingly, the user may want the ability to change the design variables for sections forward, middle, and rear sections of the prosthetic foot to affect the roll through parameters and characteristics of the prosthetic foot device.

The roll through of a prosthetic foot is defined in the gait cycle as the process from the heel-strike phase to the mid-stance phase to the toe-off phase, where the foot is no longer in contact with the ground. As the user moves through the gait cycle the tibia portion of the leg, or that section of the leg defined below the knee, rotates through in relation to the ground. The rotation of the lower leg on the theoretical ankle is notated as tibial progression or lower leg progression during the stance phase.

During the gait cycle modifying the design variables of the forward, middle, and rear sections of the prosthetic foot will have the effect of modifying the moment acting at the ankle and thus the tibial progression moment the user experiences. Modifying the design variables will also affect the ground forces on the user as they are transferred from prosthetic foot to the leg of the user.

In various embodiments, modifications to a mid-stance support with multiple design variables may have a significant impact on tibial progression between heel-strike phase, mid-stance phase through toe-off phase. The mid-stance support may comprise design variables, such as, height, width, durometer, shape, and the like. For example, a convex shape, with respect to the ground, for the mid-stance support would have the effect of lifting the user through mid-stance phase during the opposite leg swing phase, which helps the user avoid toe stub during the swing phase. The convex shape would also provide a smooth and natural tibial progression with less energy effort to overcome toe-off phase and the lower leg rotation would progress in an uninterrupted manner. The convex shape of the mid-stance support may also be changed to provide different characteristics. The orientation/placement of a radius point and thus a radius of curvature may affect the characteristic or feel of the prosthetic foot.

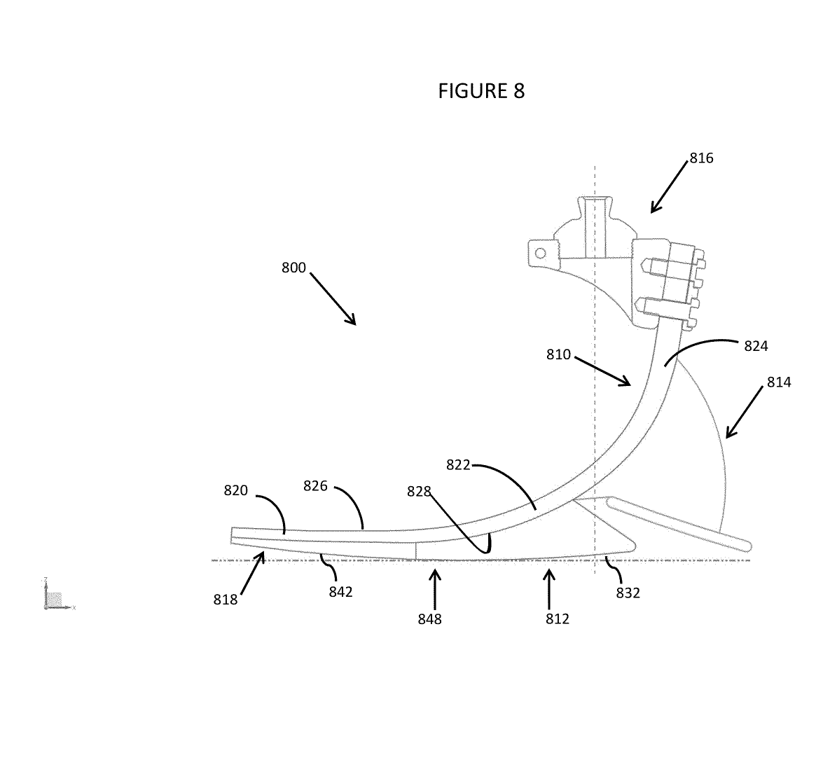

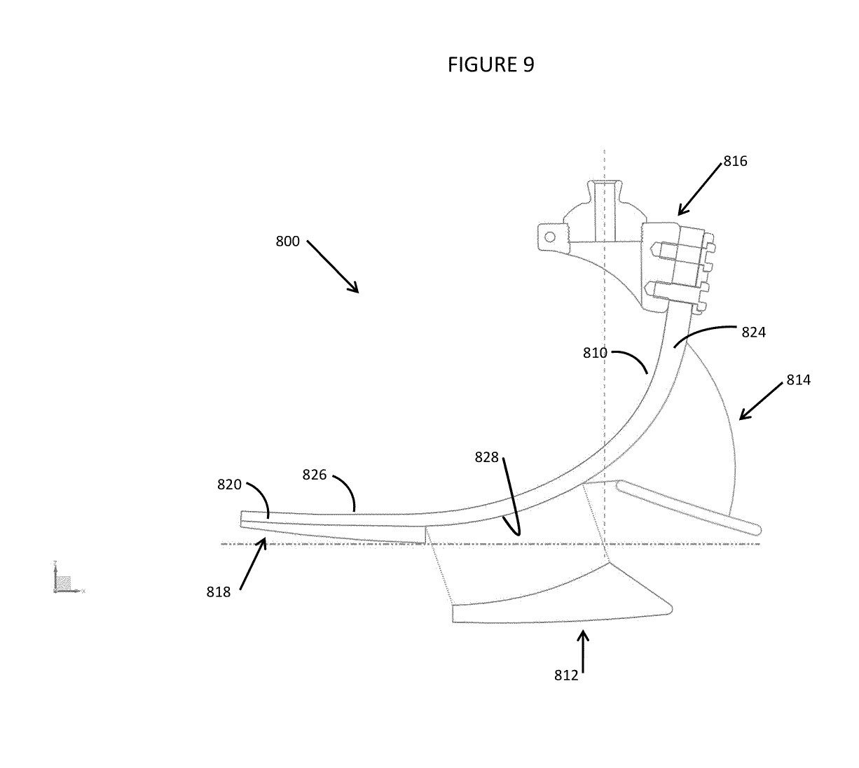

In various embodiments and with reference to FIGS. 8 and 9, a prosthetic foot 800 may comprise a resilient member 810, a mid-stance support 812, a heel support 814 and a connection point 816 attached to the resilient member 810 and configured for attachment to a user. In various embodiments, the prosthetic foot may comprise a toe piece 818.

The resilient member 810 may comprise an anterior end 820, a middle portion 822, and a posterior end 824. The resilient member 810 may comprise an arc shape, which may operate like a leaf-spring to store potential energy and carry a load when in use. The resilient member 810 may comprise an inner arc surface 826 and an outer arc surface 828.

In various embodiments, the mid-stance support 812 may be coupled to the middle portion 822 of the outer arc surface 828 of the resilient member 810. In one embodiment, the mid-stance support 812 may be removably coupled to the middle portion 822 of the outer arc surface 828 of the resilient member 810. FIGS. 8 and 9 illustrate the mid-stance support 812 in an installed position and a removed position. In the installed position, the mid-stance support 812 provides support to the prosthetic foot 800 in everyday conditions, such as standing and casual walking by reducing a deformation of the resilient member 810 to limit an amount of stored potential energy. In the removed position, shown in FIG. 9, a more aggressive toe-off is provided, which is useful in athletic endeavors, such as running, basketball, football, and the like. In various embodiments, the mid-stance support 812 may comprise different shapes to change the roll through effect of the prosthetic foot 800. In some embodiments, the mid-stance support 812 may comprise convex, concave or a combination thereof to accommodate user preferences with foot performance.

In various embodiments, the mid-stance support 812 may comprise an elastomeric material. The elastic material may comprise a natural rubber, a synthetic rubber, or various combinations of natural and synthetic rubber. The durometer of the elastomeric material may be varied to provide additional adjustment of the prosthetic foot 800. The elastomeric material of the mid-stance support 812 supports load and provides spring reaction for roll through from heel-strike phase through the mid-stance phase to the toe-off phase. The adjustable durometer of the elastomeric material allows the user to adjust the spring rate of the mid-stance support 812 based on user needs such as activity level, compliance level, weight changes, and the like. For example, in various embodiments, the durometer of the elastomeric material can be increased for users with more heel strike force, which may be caused by additional weight of the user or dynamic activity of the user. Increased heel strike force also provides greater compression of the mid-stance support 812. Thus, a user may have multiple mid-stance supports with varying durometer that are interchangeable to change the roll through characteristics of the foot based on the user's needs.

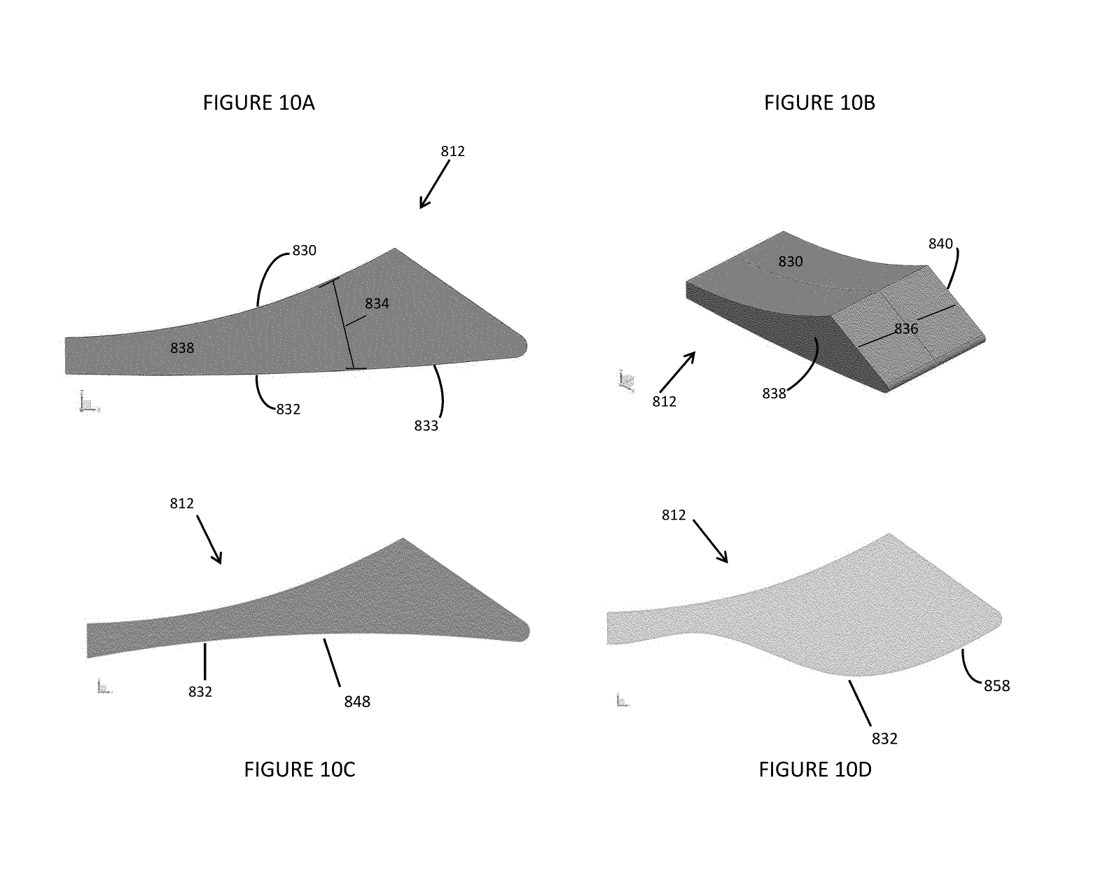

Referring now to FIGS. 8, 10A and 10B, in various embodiments, the mid-stance support 812 may comprise an upper surface 830 and a lower surface 832. The upper surface 830 of the mid-stance support 812 may be shaped to facilitate attachment to the outer arc surface 828 of the middle portion 822 of the resilient member 810. As stated above, the mid-stance support 812 may removably coupled to the resilient member 810. In one embodiment, the mid-stance support 812 may removably coupled to the resilient member 810 by adhesive or double-sided tape. In one embodiment, the mid-stance support 812 may be removably coupled to the resilient member 810 by a “slide-in” type of joint or a dovetail joint. In another embodiment, the mid-stance support 812 may be coupled to the footshell of the user (not shown), and allowed to contact the outer arc surface 828 of the middle portion 822 of the resilient member 810.

In various embodiments, the mid-stance support 812 may comprise a height 834, located between the upper surface 830 and the lower surface 832. The height 834 may be adjusted to provide more support to the mid-stance of the user during the swing though phase. For example, by increasing the height 834, the elevation of the prosthetic foot 800 of the user is raised, thus providing easier swing through of the users other foot, when the user is walking. The height 834 may be altered by different methods. In one embodiment, the height 834 may be changed by adding or removing a portion of the material through dimension 834. In one embodiment, the height 834 may be altered by changing a radius point and radius of curvature of the mid-stance support 812, as will be further discussed below. Adjusting the height 834 allows the user to experience different levels of spring support and lift during the swing through phase of the opposite leg and may also compensate for softer durometer of elastomer with more compression.

In various embodiments, the mid-stance support 812 may comprise a width 836, located between a pair of sides 838, 840. Decreasing the width 836 of the mid-stance support 812 will provide less material in contact with the outer arc surface 828 of the middle portion 822 of the resilient member 810, which lessens the dampening and load bearing capabilities of the mid-stance support 812. For example, a heavier user may be better-suited using a mid-stance support 812 with a large width, and thus a large cross-sectional area compared to a lighter user using a mid-stance support 812 with a smaller width, and cross-sectional area.

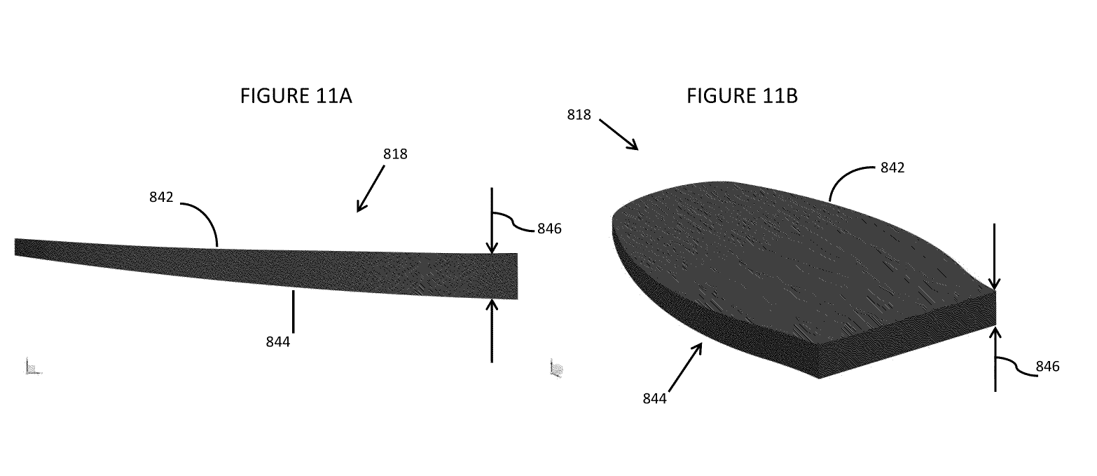

Referring now to FIGS. 8 and 11A-B, in various embodiments, the toe piece 818 may comprise an upper surface 842 and a lower surface 844. The toe piece 818 may be coupled to the outer arc surface 828 of the anterior end 820 of the resilient member 810. In one embodiment, toe piece 818 comprises approximately a constant thickness. In one embodiment, the toe piece 818 may comprise a thickness that tapers towards the front of the prosthetic foot 800. In other words, the toe piece 818 closer to the heel can be thicker than the toe piece 818 closer to the toe. Further, the adhesive bonding of the toe piece 818 can produce distributed stresses. The toe piece 818 may be bonded to the resilient member 810 in any manner discussed above or contemplated.

In various embodiments, the toe piece 818 may be removably coupled to the resilient member 810. In one embodiment, toe piece 818 may be removably coupled to the resilient member 810 by adhesive or double-sided tape. In one embodiment, toe piece 818 may be removably coupled to the resilient member 810 by a “slide-in” type of joint or a dovetail joint. In another embodiment, toe piece 818 may be coupled to the footshell of the user (not shown), and allowed to contact the outer arc surface 828 of the anterior end 820 of the resilient member 810.

In various embodiments, the toe piece 818 may comprise a height 846. As stated above, in one embodiment, the height 846 of the toe piece 818 may be constant for a uniform thickness. In another embodiment, the height 846 of the toe piece 818 may vary and the thickness of the toe piece 818 may taper towards the front of the prosthetic foot 800 to provide smooth roll through and tibial progression. In another embodiment, the height 846 of the toe piece 818 may vary and the thickness of the toe piece 818 may taper towards the rear of the prosthetic foot 800 to provide a more stable foot which resists tibial progression.

The height 846 may be adjusted to provide more support to the toe of the user. For example, by increasing the height 846, the elevation of the prosthetic foot 800 of the user is raised, thus providing increased deflection of member 810 for an increased release of energy at toe-off phase during high activity. The thickness or height of the portion of the toe piece 818 near the front of the prosthetic foot 800 may also be increased, which results in toe roll through resistance and a stable stance for a low activity individual.

In various embodiments, the toe piece 818 may comprise an elastomeric material. The elastomeric material may comprise a natural rubber, a synthetic rubber, or various combinations of natural and synthetic rubber. The durometer of the elastomeric material may be varied to provide additional adjustment of the prosthetic foot 800. In one embodiment, the lower surface 844 of the toe piece 818 and the lower surface 832 of the mid-stance support 812 create a support surface 848. In one embodiment the support surface 848 may be arc shaped to provide for greater roll through during use. The support surface 848 from the toe piece 818 and mid-stance support 812 may be changed to alter the tibial progression profile from mid-stance phase to the toe-off phase.

In one embodiment, shown in FIGS. 10A and 10B, the lower surface 832 of the mid-stance support 812 may comprise a convex lower surface 833, with respect to the ground. A convex lower surface 833 of the mid-stance support 812 allows the roll through characteristics of the prosthetic foot 800 to be altered by lifting the prosthetic foot 800 at the mid-stance to provide a bridge type of smooth transition from the heel strike to the toe-off. When the lower surface 832 is convex, the radius of curvature is above the prosthetic foot. The convex lower surface 833 of the mid-stance support 812 would have the effect of lifting the user through mid-stance phase during the opposite leg swing phase to help the user avoid toe stub during swing phase. The convex lower surface 833 may also provide a smooth and natural tibial progression with less energy effort to overcome the toe-off phase. The convex lower surface 833 may provide a smooth roll through action from heel-strike phase to toe-off phase with more lift at stance phase and constant ground contact, which users may find more comfortable for walking. Additionally, the lower leg rotation would progress in an uninterrupted manner.

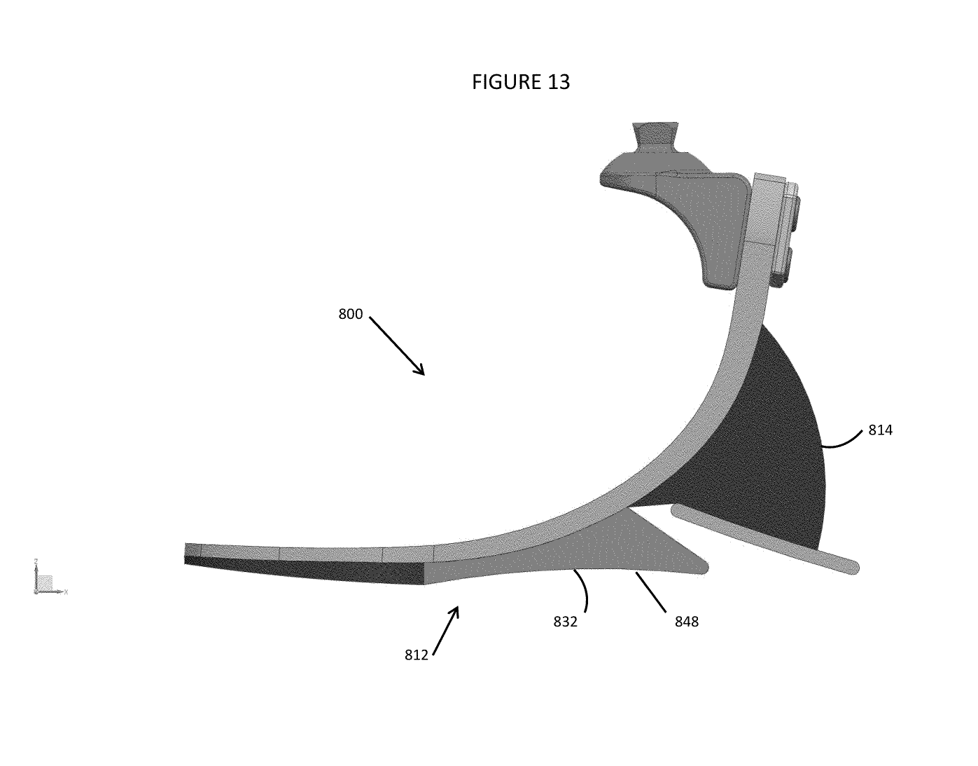

Referring now to FIGS. 10C and 13, in one embodiment, the lower surface 832 of the mid-stance support 812 may comprise a concave lower surface 848. The concave lower surface 832 of the mid-stance support 812 allows the roll through characteristics of the prosthetic foot 800 to be altered to provide a more aggressive transition from the heel-strike phase to the toe-off phase. When the lower surface 832 is concave, the radius of curvature is below the prosthetic foot. Utilizing the mid-stance support 812 with a concave lower surface 848 reduces the assistance of the mid-stance support 812 during the transition between heel-strike phase and toe-off phase. The concave lower surface 848 may assist in tibial progression after heel strike moving into flat foot stance. The concave lower surface 848 also allows an aggressive user to move more directly from heel-strike phase to toe-off phase and drives a deeper deflection of the toe providing a greater spring off the toe. The concave lower surface 848 may also diminish the effect of the mid-stance support 812, thereby providing a more aggressive heel to toe roll through with a “flat spot.”

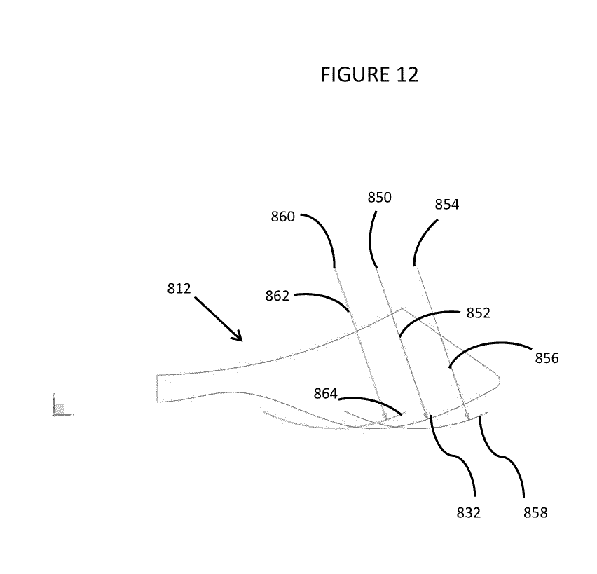

As shown in FIG. 12, the mid-stance support 812 may comprise a radius point 850 above the prosthetic foot 800. The radius point 850 is shown in a generally neutral position. The radius point 850 may be moved to alter a radius of curvature 852 of the lower surface 832 of the mid-stance support 812. The radius point 850 may be moved forward, aft, up, or down to change the shape of the lower surface 832 of the mid-stance support 812 to alter the characteristics of the prosthetic foot 800. The radius point 850 may be moved up or down to change the height 834 of the mid-stance support.

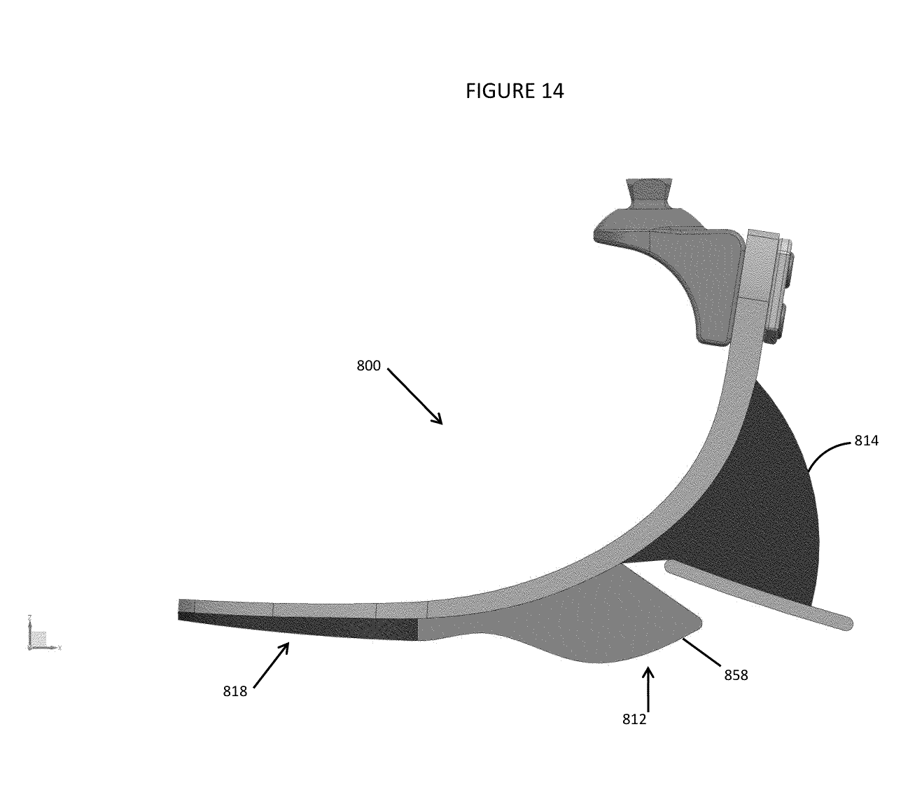

As shown in FIGS. 12, 10D and 14, the radius point 850 has been moved aft, shown by reference numeral 854, towards the rear of the prosthetic foot 800. Movement of the radius point 854 rearward causes the radius of curvature to move aft, shown by reference numeral 856, producing a larger radius peak 858 on the support surface 832, which provides a stiffer heel support 814 as the larger radius peak 858 on the rear portion of the mid-stance support 812 engages sooner from the heel-strike phase to the mid-stance phase. In use, when the user is walking and engages the heel support 814, the heel support 814 will compress and engage the mid-stance support 812 as the prosthetic foot goes from heel-strike phase to the mid-stance phase. Movement of the radius of curvature aft, shown by reference numeral 856, provides a smoother transition from heel-strike phase to the mid-stance phase. Movement of the radius point 854 rearward produces a larger radius peak 858, which lifts the leg earlier in support of heel-strike phase, and drives tibial progression forward, earlier in mid-stance phase, assisting in earlier toe-off.

Referring again to FIG. 12, the radius point 850 has been moved forward, shown by reference numeral 860, towards the front of the prosthetic foot 800. Movement of the radius point forward causes the radius of curvature to move forward, shown by reference numeral 862, shifts the radius peak forward, shown by reference numeral 864, which provides a softer heel as the rear portion of the mid-stance support 812 engages later from the heel-strike phase to the mid-stance phase. When the user is walking and engages the heel support 814, the heel support 814 will compress and engage the mid-stance support 812 as the prosthetic foot goes from heel-strike phase to the mid-stance phase. However, movement of the radius of curvature forward, shown by reference numeral 862 causes the mid-stance support 812 to engage later than the mid-stance support 812 with the radius of curvature moved rearward, shown by reference numeral 856. Movement of the radius of curvature forward provides stable heel strike with less tibial progression resulting in a more abrupt transition from heel-strike phase to the mid-stance phase and an easier transition from mid-stance phase to toe-off phase. Movement of the radius of curvature forward shifts the radius peak forward, which stabilizes the heel-strike phase, reduces the moment at the ankle, which may be advantageous for mechanical knee user stability in the phase between heel-strike phase and mid-stance phase.

In various embodiments, the radius point may also be moved up or down. Movement of the radius point up causes the radius of curvature to move upward, which provides a softer heel as the rear portion of the mid-stance support 812 engages later from the heel-strike phase to the mid-stance phase. When the user is walking and engages the heel support 814, the heel support 814 will compress and engage the mid-stance support 812 as the prosthetic foot goes from heel-strike phase to the mid-stance phase. Movement of the radius point up causes the radius of curvature to flatten out, which lowers the height 834 of the mid-stance support 812. Movement of the radius point and radius of curvature up provides a more abrupt transition from heel-strike phase to the mid-stance phase to toe-off phase.

Movement of the radius point down causes the radius of curvature to move down, which provides a stiffer heel as the rear portion of the mid-stance support 812 engages sooner from the heel-strike phase to the mid-stance phase. When the user is walking and engages the heel support 814, the heel support 814 will compress and engage the mid-stance support 812 sooner as the prosthetic foot goes from heel-strike phase to the mid-stance phase. Movement of the radius point down causes a taller radius of curvature, which raises the height 834 of the mid-stance support 812. Movement of the radius of curvature down provides a smoother transition from heel-strike phase to the mid-stance phase which may be useful when using a lower durometer rubber on the mid-stance support 812, which would compress more. The raised height 834 of the mid-stance support 812 compensates for the increased compression of the mid-stance support 812, which may be ideal for more shock absorption and greater multi-axial (inversion/eversion) effect.

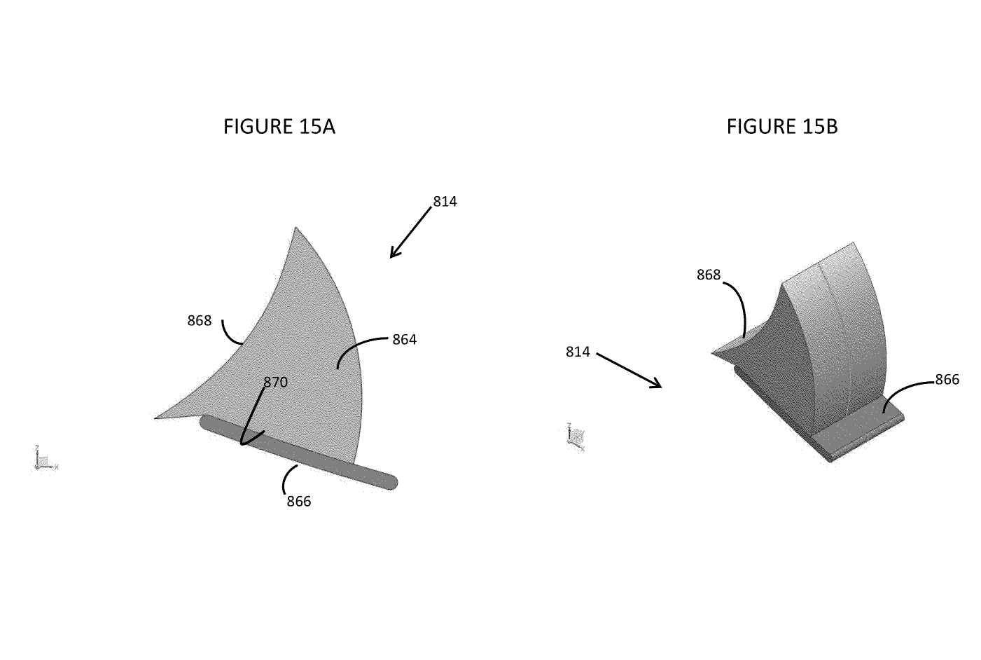

Referring now to FIGS. 8, 15A and 15B, the heel support 814 may comprise a bumper member 864 and a heel lock 866. The bumper member 864 may comprise a contact surface 868 and a lower surface 870. The heel lock 866 may be coupled to the lower surface 870 of the bumper member 864. The heel support 814 may be vertically oriented with respect to the prosthetic foot 800. The heel support 814 may act as a heel shock for absorbing force on the downward strike during the user's stride.

The contact surface 868 facilitates attachment to the resilient member 810. The contact surface 868 may be shaped to facilitated attachment to the posterior end 824 of the outer arc surface 828 of the resilient member 810. The bumper member 864 may be attached to the outer arc surface 828 of the resilient member 810 in various manners. For example, the bumper member 864 can be fixedly attached using adhesive or fasteners, such as screws. In another example, the bumper member 846 may be detachable using fasteners for replacement purposes and interchangeability and adjustability. Similar to the mid-stance support 812, the heel support 814 may be attached through double sided adhesive, hook and loop or other attachable and detachable method.

The heel lock 866 may comprise a shape that conforms with the shape of the lower surface 870 to facilitate attachment to the foot insert. In various embodiments, the shape of the lower surface 870 and heel lock 866, and thus the heel shape in contact with the ground may be convex or concave, have varying widths and durometer similar to the mid-stance support 812 and the toe piece 818.

The heel lock 866 may also comprise different angles relative to the ground. In various embodiments, the angle of the heel lock 866 with respect to horizontal may be adjusted to provide more or less heel strike reaction. For example, a greater angle may provide for more heel strike moment while a lesser angle provides less heal strike moment. This angle and moment may increase or decrease the tibial progression to mid stance. The angle of the heel strike affects the reaction forces on the foot to increase or decrease tibial progression of the foot to promote roll through at various levels.

A convex heel shape allows the user to get off the heel quicker and smoothly transition to the mid stance with less tibial resistance, while a concave heel shape may increase the strike angle of the heel strike, and advance the tibial progression to mid-stance, thereby driving the moment on the ankle to rotate the lower leg forward.

In various embodiments, the bumper member 864 can be made from an elastomeric material. The elastic material may comprise a natural rubber, a synthetic rubber, or various combinations of natural and synthetic rubber. The durometer of the elastomeric material may be varied to provide additional adjustment of the prosthetic foot 800. In one embodiment, the elastomeric material has about 80% or greater energy return. In another embodiment, the elastomeric material has about 90% or greater energy return. The bumper member 864 can be designed to behave similar to a non-linear spring, thereby allowing larger deflection of the posterior toe during the heel strike. The progressive “spring rate” may lead to a soft heel strike but does not deflect too far as the bumper member compresses.

The prosthetic foot 800 can be adjusted to accommodate a user in part by adjusting characteristics of the bumper member 864. For example, in various embodiments, the durometer of the bumper member 864 can be increased for users with more heel strike force, which may be caused by additional weight or dynamic activity. A heavier user may be better-suited using a bumper member 864 with a large cross-sectional area compared to a lighter user using a bumper member with a small cross-sectional area. Changing of durometer of the bumper member 864 to a softer heel would reduce shock during walking and relatively low activity while a stiff durometer heel on the bumper member 864 would provide a more responsive reaction of the heel of the foot during extreme athletic events.

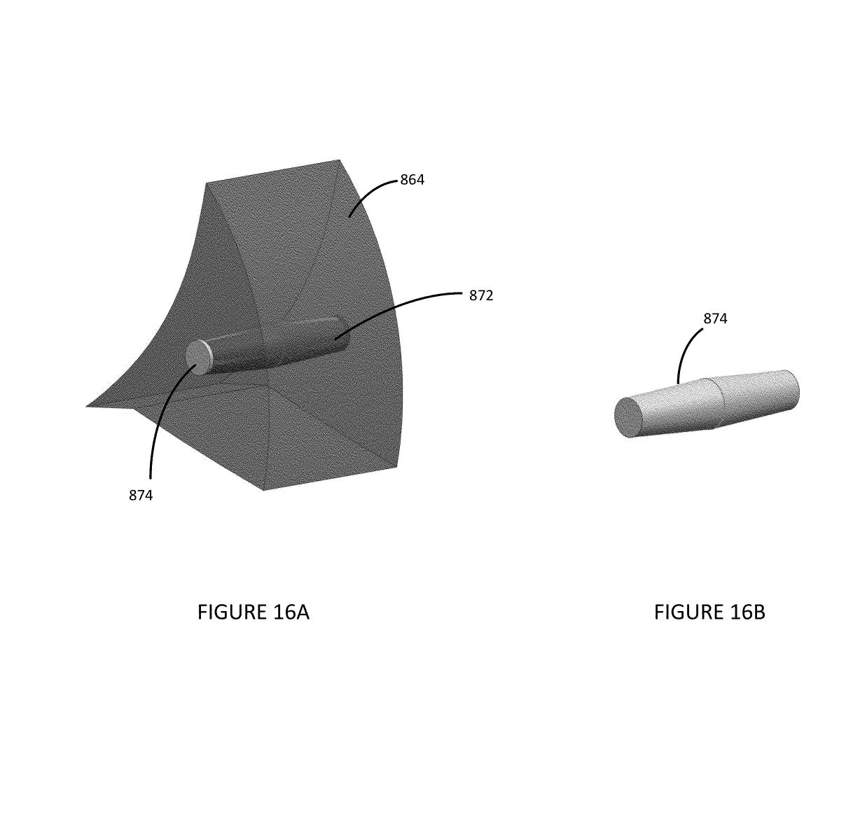

The bumper member 864 may be constructed in many shapes. In various embodiments, the bumper member 864 may have a conical, rectangular, concave, or pyramid shape. In various embodiments the bumper member 864 may have portions removed for further adjustability. For example, as shown in FIGS. 16A and 16B, a portion of the bumper member 864 may be removed to create a cavity 872 thereby providing less spring rebound and load bearing of the heel support 814. To provide greater spring rate, an insert 874 having a greater durometer than the bumper member 864 may be placed within the cavity 872, thereby increasing the spring rate and load bearing characteristics of the heel support 814. To provide less spring, an insert 874 having a lesser durometer than the bumper member 846 may be placed within the cavity 872 thereby decreasing the spring rate and load bearing characteristics of the heel support 814. The insert 874 may be made of any suitable material with varying durometer to change the stiffness and load bearing characteristics of the heel support 814. The cavity 872 and insert 874 may comprise any shape suitable to vary the support characteristics of the heel support 814. For example, a “double tapered” insert may be inserted to change the characteristic of the heel.

The resilient member 810, the mid-stance support 812, and the heel support 814 transfer energy between themselves in a natural, true foot manner. When prosthetic foot 800 is compressed, resilient member 810, the mid-stance support 812, and the heel support 814 are compressed and displaced downwardly.

The loading response during the heel-strike phase contacts the heel lock 848 and compresses bumper member 846 of the heel support 814 and resilient member, which in turn passes energy into, and causes a deflection of, the heel lock 848 of the heel support 814, which transfers energy towards the front of prosthetic foot 800 during the mid-stance phase and through the mid-stance support 812. The contact surface 844 of the mid-stance support 812 combined with the lower surface 842 of the toe piece 818 provides support from heel-strike phase through the mid-stance phase, and to the toe-off phase, which provides a more natural walking motion. Furthermore, an upward deflection of the resilient member 810 stores energy during the transition from the mid-stance phase to the toe-off phase of the gait cycle. In an exemplary embodiment, about 90% or more of the heel strike loading energy is stored and transferred to top member 120 for assisting the toe-off phase. In another exemplary embodiment, about 95% or more of the heel strike loading energy is stored and transferred to top member 120 for assisting the toe-off phase. In yet another exemplary embodiment, about 98% or more of the heel strike loading energy is stored and transferred to top member 120 for assisting the toe-off phase.

In accordance with an exemplary embodiment, the resilient members 110, 120, and 810 may be made of glass fiber composite. The glass fiber composite may be a glass reinforced unidirectional fiber composite. In one embodiment, the fiber composite material is made of multiple layers of unidirectional fibers and resin to produce a strong and flexible material. The fibers may be glass fibers or carbon fibers. Specifically, layers of fiber are impregnated with the resin, and a glass reinforcement layer may be positioned between at least two fiber layers. Typically, several layers of the unidirectional fibers or tape are layered together to achieve the desired strength and flexibility. Further, in various embodiments the layers of unidirectional fibers or tape may be oriented at various angles.

The invention has been described with reference to specific exemplary embodiments. Various modifications and changes, however, may be made without departing from the scope of the present invention. The description and figures are to be regarded in an illustrative manner, rather than a restrictive one and all such modifications are intended to be included within the scope of the present invention. Accordingly, the scope of the invention should be determined by the generic embodiments described and their legal equivalents rather than by merely the specific examples described above. For example, the steps recited in any method or process embodiment may be executed in any order, unless otherwise expressly specified, and are not limited to the explicit order presented in the specific examples. Additionally, the components and/or elements recited in any apparatus embodiment may be assembled or otherwise operationally configured in a variety of permutations to produce substantially the same result as the present invention and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

The present invention has been described above with reference to a preferred embodiment. However, changes and modifications may be made to the preferred embodiment without departing from the scope of the present invention. These and other changes or modifications are intended to be included within the scope of the present invention, as expressed in the following claims.

1. A prosthetic foot comprising:
a resilient member comprising an anterior end, a middle portion, and a posterior end;
a mid-stance support coupled to a lower surface of the middle portion of the resilient member; and
a heel support coupled to a lower surface of the posterior end of the resilient member.

2. The prosthetic foot of claim 1, wherein the mid-stance support is removably coupled to the resilient member.

3. The prosthetic foot of claim 1, wherein the mid-stance support comprises an elastomeric material configured to be adjustable in durometer.

4. The prosthetic foot of claim 1, wherein the mid-stance support comprises an elastomeric material configured to be adjustable in height.

5. The prosthetic foot of claim 1, wherein the mid-stance support comprises a concave-shaped lower surface.

6. The prosthetic foot of claim 1, wherein the mid-stance support comprises a convex-shaped lower surface.

7. The prosthetic foot of claim 6, wherein the convex-shaped lower surface comprises a radius peak located at a rear portion of the mid-stance support configured to provide a smooth transition from the heel-strike phase to mid-stance phase.

8. The prosthetic foot of claim 6, wherein the convex-shaped lower surface comprises a radius peak located at a forward portion of the mid-stance support configured to provide a heel strike with less tibial progression and an abrupt transition from the heel-phase to mid-stance phase.

9. The prosthetic foot of claim 1, wherein the mid-stance support comprises a lower surface with a radius point located above the foot.

10. The prosthetic foot of claim 1, wherein the mid-stance support comprises a lower surface with a radius point located below the foot.

11. The prosthetic foot of claim 1, wherein the heel support comprises an elastomeric bumper and a heel lock having an angled surface.

12. The prosthetic foot of claim 11, wherein the elastomeric bumper is configured to be adjustable in durometer.

13. The prosthetic foot of claim 11, wherein the angled surface is configured to be adjustable to provide increased heel strike.

14. The prosthetic foot of claim 1, further comprising a toe piece coupled to a lower surface of the anterior end of the resilient member.

15. The prosthetic foot of claim 14, wherein the toe piece and the mid-stance support form a support surface.

16. The prosthetic foot of claim 15, wherein when the support surface is configured to provide support from the heal-strike phase to the toe-off phase.

17. The prosthetic foot of claim 11, wherein the resilient member and the angled surface of the heel support are capable of storing energy during deflection to propel the user forward.

18. A prosthetic foot comprising:
a resilient member comprising an outer arced surface;
a toe piece coupled to an anterior portion of the outer arced surface of the resilient member;
a mid-stance support removably coupled to a middle portion of the outer arced surface of the resilient member; and
a heel support coupled to a posterior portion of the outer arced surface of the resilient member.

19. The prosthetic foot of claim 18, wherein the mid-stance support comprises an elastomeric material configured to be adjustable in durometer.

20. The prosthetic foot of claim 18, wherein the mid-stance support comprises an elastomeric material configured to be adjustable in height.

21. The prosthetic foot of claim 18, wherein the mid-stance support comprises a convex-shaped lower surface.

22. The prosthetic foot of claim 18, wherein the mid-stance support comprises a concave-shaped lower surface.

23. The prosthetic foot of claim 21, wherein the convex-shaped lower surface comprises a radius peak located at a rear portion of the mid-stance support.

24. The prosthetic foot of claim 21, wherein the convex-shaped lower surface comprises a radius peak located at a rear portion of the mid-stance support.

25. The prosthetic foot of claim 18, wherein the heel support comprises an elastomeric bumper and a heel lock having an angled surface.

26. The prosthetic foot of claim 25, wherein the elastomeric bumper is configured to be adjustable in durometer.

27. The prosthetic foot of claim 25, wherein the angled surface is configured to be adjustable to provide increased heel strike.

28. The prosthetic foot of claim 25, wherein the resilient member and the angled surface of the heel support are capable of storing energy during deflection to propel the user forward.

29. The prosthetic foot of claim 18, wherein the resilient member comprises a glass fiber.
